(19) United States
(12) Patent Application Publication
(10) Pub. No.: US 2012/0252062 A1
(43) Pub. Date: Oct. 4, 2012

(54) Method for diagnosing and/or predicting the development of neurodegenerative diseases

(75) Inventors: Saskia Biskup, Stuttgart (DE); Natalja Funk, Tubingen (DE)

(73) Assignee: EBERHARD-KARLS-UNIVERSITAT TUBINGEN UNIVERSITATSKLINIKUM, Tubingen (DE)

(21) Appl. No.: 13/416,203

(22) Filed: Mar. 9, 2012

Related U.S. Application Data
(63) Continuation of application No. PCT/EP2010/063406, filed on Sep. 13, 2010.

(30) Foreign Application Priority Data
Sep. 11, 2009 (DE) 102009042160

(51) Int. Cl. C12Q 1/06

(52) U.S. Cl. 435/39

(57) ABSTRACT

The present invention relates to a method for diagnosing and/or predicting the development of neurodegenerative diseases; in the method, white blood cells are isolated and enriched or cultivated for forming colony-forming units, wherein CFU-M and other CFU are formed. Subsequently, the relative number of CFU-M formed in the previous cultivation(/enrichment step relative to the other CFUs formed are determined.

### CROSS REFERENCE TO RELATED APPLICATIONS

This application is a continuation of international patent application PCT/EP2010/063406, filed on Sep. 13, 2010 designating the U.S., which international patent application has been published in German language and claims priority from German patent application DE 10 2009 042 160.2, filed on Sep. 11, 2009. The entire contents of these priority applications are incorporated herein by reference.

### BACKGROUND OF THE INVENTION

The present invention relates to a method for diagnosing and/or predicting the development of neurodegenerative diseases.

Neurodegenerative diseases or disorders are characterized by slow, unrelenting death of nerve cells. Human neurodegenerative diseases include, among others: amyotrophic lateral sclerosis (ALS), tauopathies, e.g. Alzheimer's disease, trinucleotide diseases, e.g. Huntington's chorea (chorea), prion diseases, e.g. Creutzfeldt-Jakob disease, and synucleopathies, e.g. Parkinson's disease. In particular, Alzheimer's disease and Parkinson's disease are a frequent cause of dementia and consequent need for care in old age.

To date, there is no known test on the market, with which neurodegenerative diseases, in particular those that are not caused genetically, can be established early and beyond doubt. However, early recognition of persons at risk of developing a neurodegenerative disease would be desirable, because late diagnosis of a neurodegenerative disease means that a large proportion of the nerve cells in the affected person/in the patient have already degenerated, and therapy therefore starts too late. With early identification of patients at risk and early commencement of therapy, the loss of nerve cells in these patients might be prevented, or at least slowed down early.

DE 10 2007 024 382 A1 describes a method of diagnosis of a neurodegenerative disease in which the level of expression of certain genes is investigated in a biopsy sample from a patient. This method has the disadvantage that taking of the biopsy can be unpleasant and painful for the patient being investigated, and that a reliable indication of the risk of developing a neurodegenerative disease is not obtained. Furthermore, the costs of equipment as well as other costs are high, because as a rule a reliable diagnosis requires the detection of several genes.

Moreover, in the prior art, various mutations of the LRKK2 gene are known, which are regarded as genetic markers for familial Parkinson's disease. However, with these markers it is only possible to determine the risk of developing a specific disease, and moreover the markers also are only familial markers.

### SUMMARY OF THE INVENTION

Against this background, it is an object of the present invention to provide a new method or new markers, with which the disadvantages of the prior art can be overcome and the risk of developing neurodegenerative diseases can be predicted rapidly and reliably, with good tolerance by the person being investigated

According to one aspect of the invention, there is provided a method for diagnosing and/or predicting the development of neurodegenerative diseases, wherein the method has the steps stated in the claims.

In developing the method according to the invention, the inventors took into account the fact that, via the blood and the lymphatic system, there is constant exchange between the nerve cells of the brain on the one hand and the cells of the immune system on the other hand. Neurodegenerative diseases lead, among other things, to the death of individual nerve cells. This can have various causes. Dying nerve cells lead in their turn to the release of signal substances, which attract brain-resident macrophages and other cells of the immune system. The inward migration of immune cells in its turn causes nerve cell death, so that a reaction develops that is no longer controllable, and finally forms the basis for the slowly advancing neurodegenerative disease. Furthermore, signal substances are released into the blood and the lymphatic system. These signal substances also exert an action on white blood cells in the periphery and on their precursor cells, for example in the bone marrow. This may, among other things, initiate the proliferation and differentiation of certain white blood cells. This increase in white precursor cells is utilized by the method according to the invention.

The method according to the invention has the following consecutive steps:
a) isolation of white blood cells from a blood sample from a person to be investigated;
b) enrichment and/or cultivation of the white blood cells isolated in step a), for example in a medium, for forming various colony-forming units (CFUs); and
c) determination and evaluation of the relative number of the CFU-M formed in step b) relative to the other CFUs formed.

According to another aspect of the invention, there is provided a method for using a CFC assay and a medium containing methylcellulose, in which white blood cells can be cultured with formation of various colonies, for diagnosing and/or predicting the development of neurodegenerative diseases.

According to another aspect of the invention, there is provided a kit, which contains a medium containing methylcellulose and instructions for carrying out the method according to the invention.

With the novel method, or the novel use and the novel kit, it is now possible for the first time to determine, by investigating a blood sample, or by investigating the colony formation of the white blood cells isolated therefrom, whether the person from whom the blood sample to be investigated was obtained does or does not have a risk of developing a neurodegenerative disease. Thus, the method according to the invention is used for investigating the proportion of CFU-M in the total number of colonies obtained after cultivation of the white blood cells. The inventors of the present application have on the one hand shown that in parkinsonian patients the proportion of CFU-M formed is greater than in healthy controls. Furthermore, it was shown by means of the method according to the invention that the proportion of CFU-M formed is also greater in persons bearing mutations in a particular gene, which serves as a marker for familial Parkinson's disease, than in controls. Therefore the presence of a higher proportion of CFU-M colonies allows a conclusion to be drawn regarding a person's risk of developing a neurodegenerative disease.

As in the prior art, “CFU” and “CFC” have the following meanings: CFC (colony-forming cells) are stem cells/precursor cells, which in contrast to pluripotent stem cells are further differentiated and are established on particular cell differentiation lines. In the colony-forming unit-culture (CFU-C) assay they can, by adding colony-stimulating factors (CSF), be stimulated to form colonies of this cell line. The cells of the resultant colonies can then be identified on the basis of their morphology and certain surface markers, and the precursors are then divided into so-called CFU (colony forming units): thus, a distinction is made between the following—which come under the term “CFU” in the present text—for example CFU-Bas (“basophil”; precursor cell of hematopoietic cells with recognition marker CD34); CFU-E (“erythrocyte”; precursor cell of erythrocytes); CFU-Eo (Eo-CFC “eosinophil”; precursor of eosinophilic granulocytes); CFU-G (G-CFC, “granulocyte”; precursor cell of granulocytes); CFU-GEMM (“granulocyte, erythrocyte, megakaryocyte, macrophage”; precursor cell of granulocytes, erythrocytes, megakaryocytes, macrophages); CFU-GM (GM-CFC, “granulocyte, macrophage”; precursor cell of granulocytes, macrophages); CFU-M (M-CFC, “macrophage”; precursor cell of macrophages); CFU-MEG (“megakaryocyte”; precursor cell of megakaryocytes).

Hematopoietic stem cells and precursor cells occur not only for example in the bone marrow, but also in the peripheral blood. By isolating white blood cells from the peripheral blood, precursor cells are therefore also isolated, which in certain cultivation conditions proliferate and differentiate and form the aforementioned colonies. In other words, from the colony formation in the CFC assay it is therefore possible to count the hematopoietic precursor cells in a sample.

Thus, in the method according to the invention, first the white blood cells from a blood sample that has been obtained, which advantageously contains fresh blood (heparinized peripheral blood), are isolated from the other blood constituents, for example by gradient centrifugation or by means of antibodies, and cultured in a medium that allows colony formation. The culture time is at least 10 days, preferably 14 to 20 days. After cultivation, the colonies formed are counted and the values for particular individual CFUs are compared.

The inventors have now shown in their own experiments that both patients with Parkinson's, and persons who bear the above-mentioned mutation in the LRRK2 gene (“leucine-rich repeat kinase 2” gene) (but have not yet displayed any signs of Parkinson's), displayed values with respect to CFU formation that differed from those of control samples. In one embodiment of the method according to the invention it is therefore preferred if in step c) the relative number of at least two of the following CFUs formed in step b) is determined and compared, namely CFU-G (CFU-granulocyte) and CFU-M (CFU-macrophage). In another embodiment, additionally the relative number of CFU-GM (CFU-granulocyte/macrophage) is also determined.

By comparing or matching the values for the relative number of the various CFUs it is possible to determine whether the value for the CFU-M is higher than for example for the CFU-G.

On the whole it was found that for parkinsonian patients, or for persons at risk of developing Parkinson's, the relative number of CFU-M was higher than for the control samples; therefore in one embodiment of the method according to the invention it is preferred if in each case the relative number of CFU-M and CFU-G is determined and these values for patients and healthy subjects are compared. An increased value for the CFU-M is associated with risk of developing neurodegenerative diseases.

In the present text “relative number” denotes the proportion of a particular form of colony relative to the total number of colonies formed.

The blood sample is moreover preferably fresh, and has been obtained before-hand from a person who is to be investigated with respect to the risk of developing a neurodegenerative disease. It is to be understood that the person or the patient is a human being, and sex and age and physical condition do not play any role provided no diseases are present (a cold, infection etc.).

In the present text—as also in the relevant technical field—white blood cells or white blood corpuscles (leukocytes) are to be understood as the nucleated blood cells with defense function, and comprise granulocytes, lymphocytes and monocytes.

In a refinement of the method according to the invention, therefore in step c) a certain relative number of CFU-M is associated with the presence and/or the course and/or the severity and/or the prediction of neurodegenerative diseases.

In particular it is preferred if a number of CFU-M colonies of >25%—based on the total number of colonies—is associated with the presence and/or course and/or the severity and/or the prediction of neurodegenerative diseases.

It is to be understood that, in the context of the present invention, values also may be considered that are slightly below 25%, but still within the understanding of a person skilled in the art on reading the invention and taking account any errors in counting, are still within the scope of the invention.

In yet another a refinement of the method according to the invention it is preferred if the medium used in step b) for culture contains methylcellulose or other gelatinous substances.

With methylcellulose or other gelatinous substances, for example agarose, a semi-solid matrix forms, in which the cells are cultivated. Advantageously the cultivation medium contains methylcellulose, but it is to be understood that also any other substance that is suitable for forming a semi-solid matrix and for the cultivation of cells therein can be used.

The invention further relates to a method for using of a CFC assay for diagnosing and/or predicting the development of neurodegenerative diseases.

CFC assay (colony forming cell (CFC) assay), which is also called methylcellulose assay, means in the present text, as also in the prior art, an in-vitro assay, which is based on the ability of the hematopoietic precursor cells/precursors, to proliferate and to differentiate in colonies in a semi-solid medium (with cytokine stimulation). The colonies formed can then be counted with respect to their morphology. Thus, according to the invention, the CFC assays known per se in the prior art and available can be used for diagnosing and/or predicting the development of neurodegenerative diseases.

Therefore the present invention also relates to the use of or rather a method for using a medium containing methylcellulose in the cultivation of white blood cells for diagnosing and/or predicting the development of neurodegenerative diseases, and a kit containing a medium containing methylcellulose and instructions for carrying out the method according to the invention.

The inventors have shown here for the first time that by using a medium containing methylcellulose and the CFC assay to be applied with this medium, a means is provided with which, by applying the method according to the invention, it is possible to determine a person's risk of developing neurodegenerative diseases.

It is self-evident that the features described above and yet to be explained below can be used not only in the combination stated in each case, but also in other combinations or alone, while remaining within the scope of the present invention.

### BRIEF DESCRIPTION OF THE DRAWINGS

The invention is explained in more detail in the following description of the examples or examples of application and on the basis of the drawings, which show:

FIG. 1 a schematic review of hematopoiesis;

FIG. 2 a review of the distribution of the various colonies formed in the CFC assay (culture medium with erythropoietin (Epo)) from samples from controls; bar chart (A) and microscopic morphology of the respective clones (B);

FIG. 3 the distribution of the clones formed in the CFC assay (culture medium without Epo) in samples from controls (A) and in samples from parkinsonian patients (B), in each case shown as a bar chart;

FIG. 4 a diagram showing the relative number (in %) of CFU-M of various human populations, including carriers of LRRK2 mutations and parkinsonian patients; and

FIG. 5 a schematic review of the steps of one embodiment of the method according to the invention.

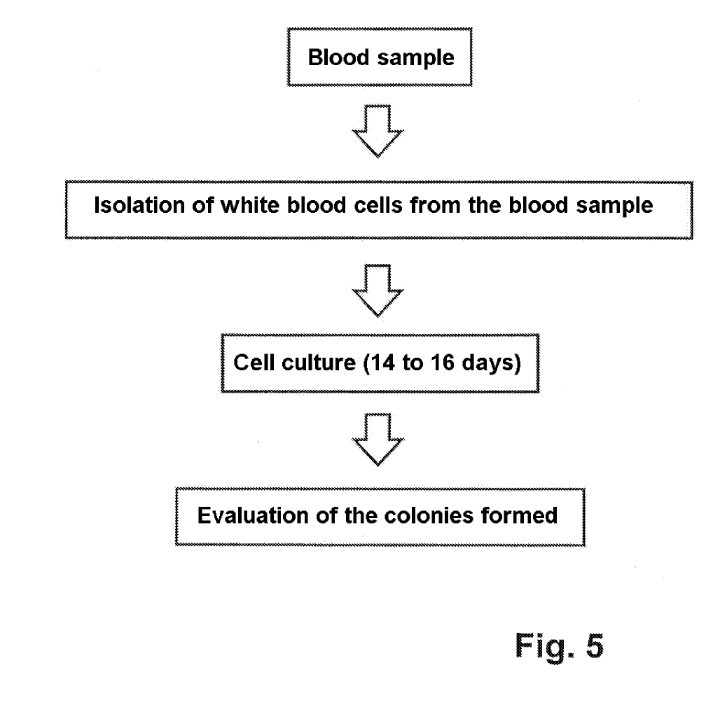

### DESCRIPTION OF PREFERRED EMBODIMENTS

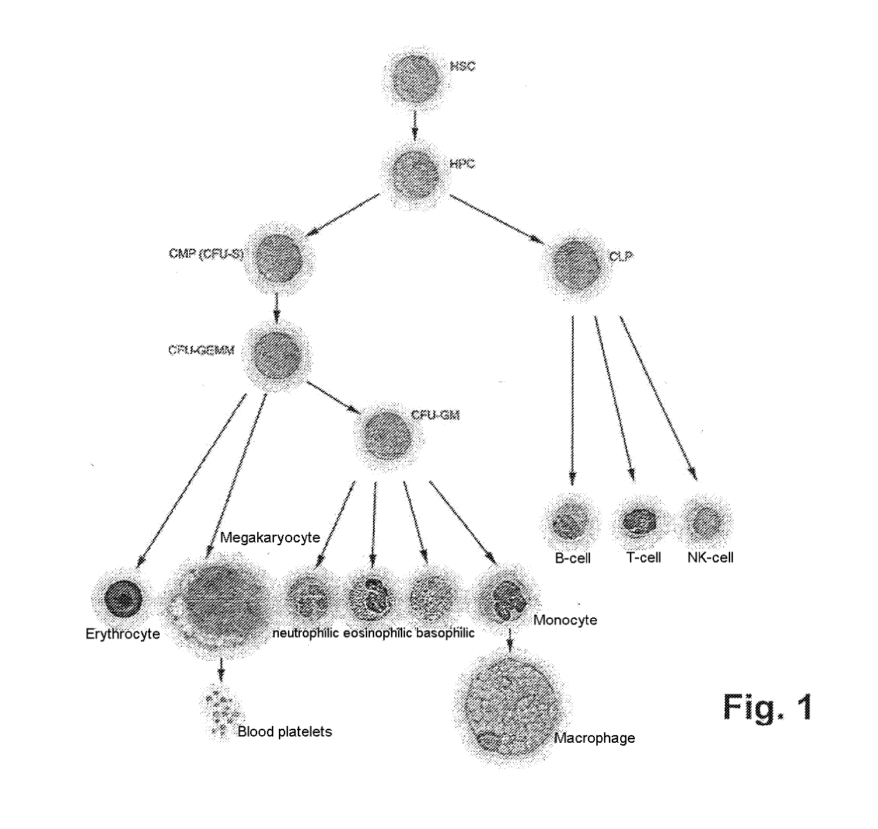

A review of hematopoiesis in humans is shown schematically in FIG. 1, where the abbreviations used in FIG. 1 have the following meanings: HSC: hematopoietic stem cells; HPC: hematopoietic precursor cells; CMP(CFU-S): common myeloid precursor; CLP: common lymphoid precursor; CFU-GEMM: colony forming unit granulocyte erythroid megakaryocyte macrophage, mixed colonies; CFU-GM: colony forming unit granulocyte macrophage. Hematopoiesis is a cell division and maturation process, which produces blood cells. The starting point of hematopoiesis is the pluripotent, undifferentiated hematopoietic stem cell (see FIG. 1: HSC), which produces precursors (or precursor cells), which cannot themselves be renewed and only bring a specialized cellular type to maturation. The immature precursor cells can circulate in the blood and can settle again in the bone marrow. The regulation of hematopoiesis is dependent on environmental factors or is humoral (e.g. through cytokines, hormones, chalones, erythropoietin). Moreover, starting from the common myeloid precursor CMP there is formation—via the CFU-GEMM intermediate—of neutrophilic, eosinophilic and basophilic granulocytes (via the further precursor CFU-GM), and erythrocytes, megakaryocytes and monocytes.

### Example

### Investigation of Clone Distribution in Control Samples and in Cultivation with EPO

In preliminary tests, samples from controls of different sex and age were investigated with respect to the distribution of the clones after cultivation of the white blood cells from these samples in a medium with erythropoietin. For this, the white blood cells are obtained after gradient centrifugation, washed, counted and taken up in a medium containing methylcellulose and cytokines (Epo (erythropoietin), SCF (stem cell factor), GM-CSF (granulocyte-macrophage colony-stimulating factor), IL-3 (interleukin-3) (all R & D Systems, Minneapolis, USA).

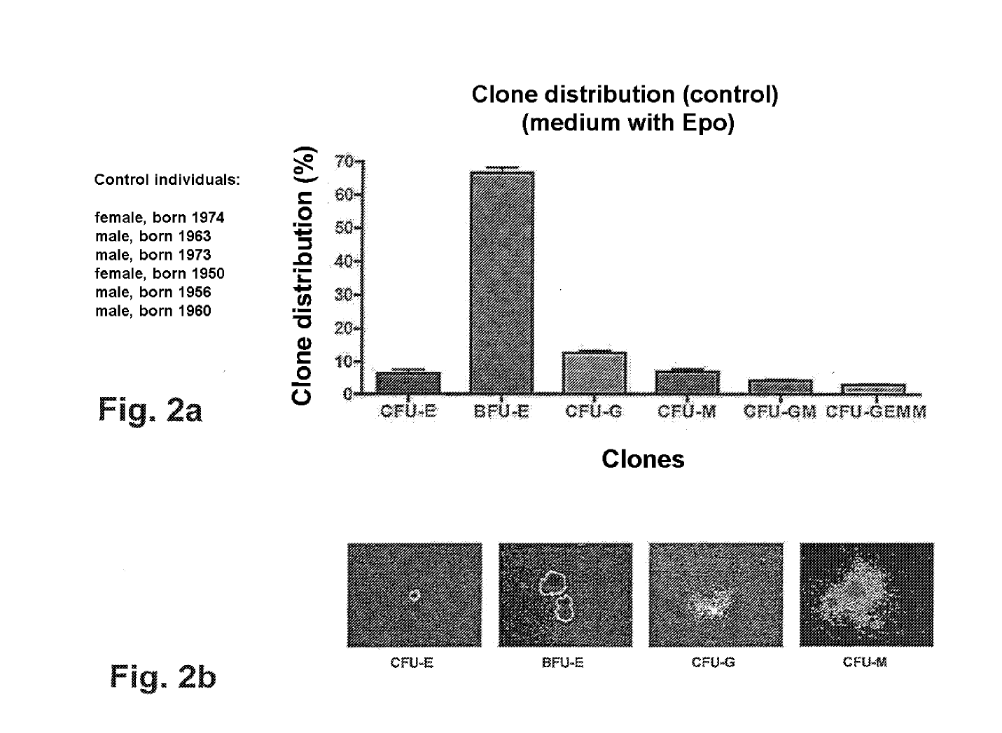

After incubation for 14 days, the distribution was as shown in FIG. 2: FIG. 2A shows the results in a bar chart, and the corresponding images of the respective clones in the photographs below the bar chart (FIG. 2B), below the respective clone. It was found that the percentages of the various precursor cells (CFU-E, BFU-E, CFU-G, CFU-M, CFU-GM, CFU-GEMM) barely differed for the controls, regardless of sex and age.

Investigation of the Distribution of Clones in Control Samples and in Cultivation without EPO

In the next tests, samples from healthy controls and samples from patients with Parkinson's disease were investigated. For this, in each case 10 ml heparinized peripheral blood from the subjects/patients was in each case separated by Ficoll density gradient centrifugation, and a cell culture was set up with the white blood cells that were separated. A commercially available medium that contained methylcellulose and other additives (SCF, GM-CSF, IL-3) was used for this (R & D Systems, Minneapolis, USA). The cell culture was cultured in an incubator for 14 days, and the cells or colonies formed were then counted.

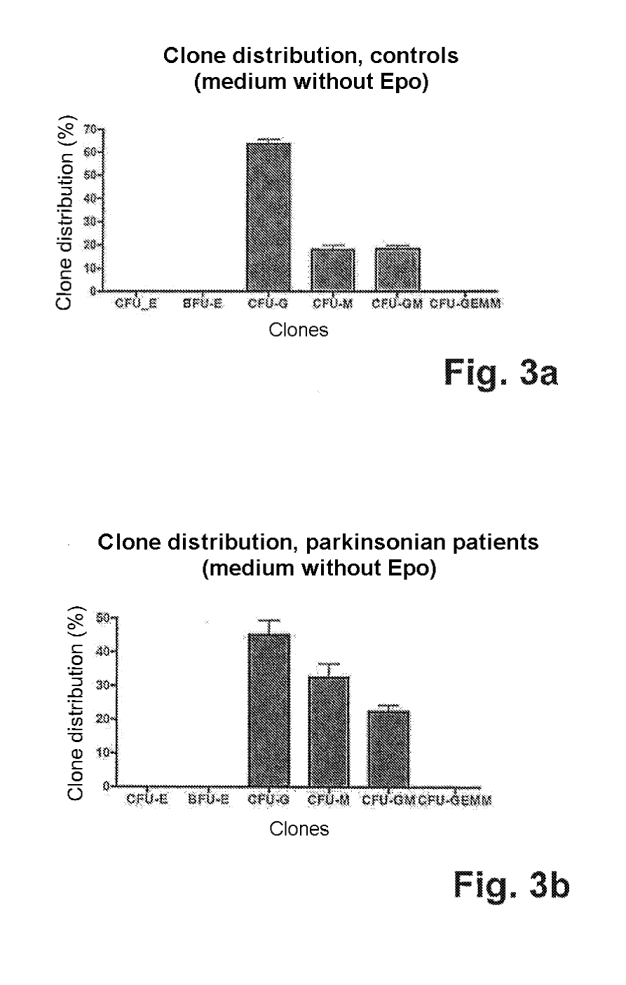

It was found that, starting from the samples from the controls, approx. 63% CFU-G, approx. 18% CFU-M and approx. 19% CFU-GM were formed (see FIG. 3A). For the samples from the patients with Parkinson's (see FIG. 3B) the distribution found was approx. 45% CFU-G, approx. 33% CFU-M and approx. 22% CFU-GM, and therefore shows a definite shift of colony formation towards CFU-M.

Investigation of Samples from Persons with LRRK2 Mutation

In further tests, samples were investigated from persons bearing various mutations in the LRRK2 gene. Mutations in the LRRK2 gene (leucine-rich repeat kinase 2 gene) are, according to the latest findings, biomarkers for familial Parkinson's disease; so far the LRRK2 mutations G20192, Q930R, and L1114L have been identified.

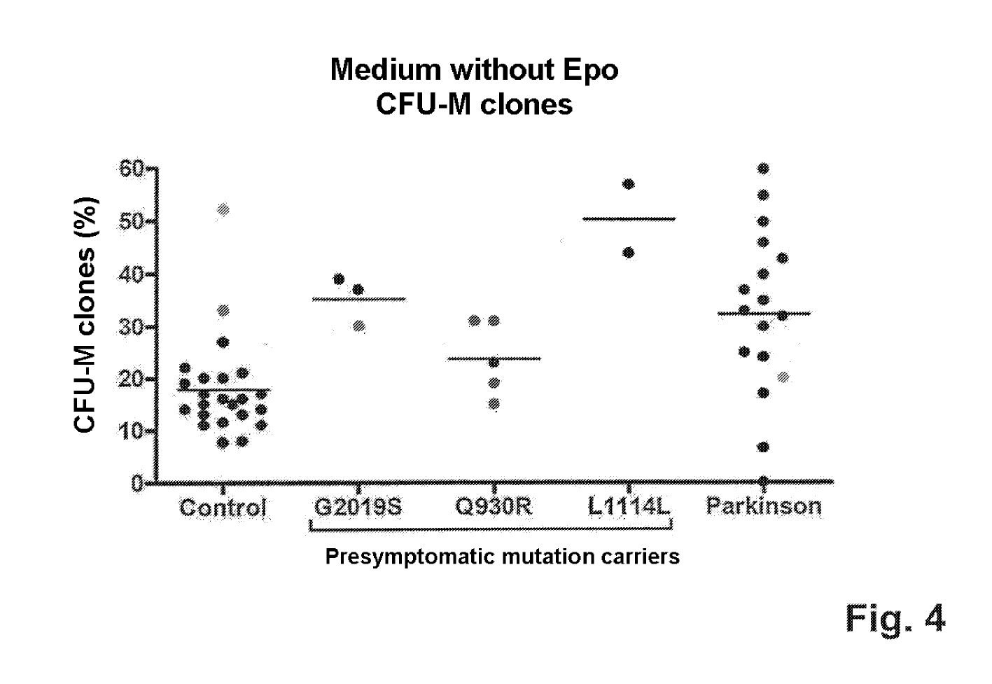

The test procedure was as described above. The colonies formed from samples from persons with these mutations were compared with controls and with those from parkinsonian patients (see FIG. 4). The previous findings for parkinsonian patients were confirmed, namely that the relative number of CFU-M in carriers of the mutations and in parkinsonian patients was on average higher than for the controls.

Therefore the inventors were able to show that the method is a suitable means for obtaining information about the risk of developing neurodegenerative diseases.

The blood samples were obtained in the course of a scientific study at the Department of Neurodegeneration of the Hertie Institute for Clinical Brain Research, Tubingen. An ethics application was in place.

The blood (approx. 10 ml) was diluted 1:1 with HBSS (Hank's Balanced Salt Solution, Invitrogen, Carlsbad, USA) and stratified by means of a 15 ml Ficoll-Paque Plus (GE Healthcare). By centrifugation for 30 min at 400×g, white blood cells are obtained, these are washed 2× with 20 ml HBSS each time, taken up in 10 ml IMDM (Iscove's Modified Dulbecco's Medium, Invitrogen) and counted. For further cultivation of the white blood cells obtained, media containing methylcellulose from R&D Systems were used (Human Methylcellulose Complete Media and Human Methylcellulose Complete Media without Epo). Approximately 500 000 cells/ml of medium were plated out. The cells were cultured in 3.5 cm cell culture dishes (BD Biosciences Falcon, San Jose, USA) at 37° C., 5% CO₂ and high humidity. On day 15 after plating out, the colonies were evaluated by optional microscopy and the percentages of the individual clone populations were calculated.

What is claimed is:

1. A method for diagnosing and/or predicting the development of neurodegenerative diseases, wherein the method has the following steps:
a) isolation of white blood cells from a blood sample from a person to be investigated;
b) enrichment and/or cultivation of the white blood cells isolated in step a) for forming various colony-forming units (CFUs), wherein CFU-M and other CFUs are formed, and
c) determination and evaluation of the relative number of CFU-M formed in step b) relative to the other CFUs formed.

2. The method as claimed in claim 1, wherein step b) is carried out in a medium for forming various colony-forming units (CFUs).

3. The method as claimed in claim 1, wherein in step c) the relative number of at least two of the following CFUs formed in step b) is determined and compared, namely CFU-G (CFU-granulocyte) and CFU-M (CFU-macrophage).

4. The method as claimed in claim 3, wherein additionally the relative number of CFU-GM (CFU-granulocyte/macrophage) is determined.

5. The method as claimed in claim 1, wherein the determination of a relative number of CFU-M that is higher than the relative number of CFU-G is associated with the presence and/or the course and/or the severity and/or the prediction of neurodegenerative diseases.

6. The method as claimed in claim 1, wherein in step c) a certain relative number of CFU-M is associated with the presence and/or the course and/or the severity and/or the prediction of neurodegenerative diseases.

7. The method as claimed in claim 6, wherein a number of CFU-M colonies of at least approx. 25% is associated with the presence and/or course and/or the severity and/or the prediction of neurodegenerative diseases.

8. The method as claimed in claim 1, wherein the medium used in step b) for culture contains methylcellulose.

9. The method as claimed in claim 1, wherein the white blood cells are isolated from the blood sample by density-gradient centrifugation or by purification by means of specific antibodies.

10. The method as claimed claim 1, wherein the white blood cells isolated are cultured for a period of at least 10 days, preferably 14 days.

11. A method for diagnosing and/or predicting the development of neurodegenerative diseases, wherein the method comprises the step of employing a CFC assay.

12. A method for diagnosing and/or predicting the development of neurodegenerative diseases, wherein the method comprises the step of using a medium containing methyl-cellulose in the cultivation of white blood cells.

13. A kit containing a medium containing methylcellulose and instructions for carrying out the method of claim 1.
